(19) United States
(12) Patent Application Publication
(10) Pub. No.: US 2022/0218818 A1
(43) Pub. Date: Jul. 14, 2022

(54) SMALLPOX VACCINE AND STEM CELLS FOR TREATMENT OF DISEASE

(71) Applicant: IMMUNOLUX INTERNATIONAL CORP., Highland, CA (US)

(72) Inventor: Aladar SZALAY, Highland, CA (US)

(21) Appl. No.: 17/615,661

(22) PCT Filed: Jun. 2, 2020
(86) PCT No.: PCT/US20/35737

§ 371 Date: Dec. 1, 2021

Related U.S. Application Data
(60) Provisional application No. 63/005,048, filed on Apr. 3, 2020.
(60) Provisional application No. 62/877,978, filed on Jul. 24, 2019.
(60) Provisional application No. 62/856,582, filed on Jun. 3, 2019.

(51) Int. Cl. A61K 39/275; A61P 29/00; A61P 31/12

(52) U.S. Cl. CPC A61K 39/275; A61K 2039/525; A61P 31/12; A61P 29/00

(57) ABSTRACT

Described herein are methods and compositions for treating an inflammatory disease or infectious disease in a subject in need thereof by administering to the subject a poxvirus and a stem cell, wherein the disease is not a cancer. The disease may be, for example, a chronic inflammatory disease (e.g., an autoimmune disease).

### BACKGROUND

Inflammatory diseases, for example autoimmune diseases, are caused by chronic inflammation in a subject. These diseases can cause symptoms ranging from mild discomfort to severe reactions, and even death.

Infectious diseases are caused by organisms, such as bacteria, viruses, fungi or parasites. While infections are often treated with antibiotics, antivirals, antifungals, antiprotozoals, and antihelminthics, pathogens are becoming increasingly resistant to these drugs. Other pathogens have no known treatments.

New methods of treating these diseases are needed.

### BRIEF SUMMARY OF THE INVENTION

Described herein are methods and compositions for treating a disease in a subject in need thereof by administering to the subject a poxvirus and a stem cell, wherein the disease is not a cancer.

In an aspect, a method for treating a chronic inflammatory disease in a subject is provided. The method includes administering to the subject a poxvirus, wherein the disease is not a cancer. In embodiments, the poxvirus is administered in a therapeutically effective amount, e.g., at an amount sufficient to treat the chronic inflammatory disease.

In an aspect, a method for treating an infectious disease and/or a symptom thereof in a subject is provided. The method includes administering to the subject a poxvirus. In embodiments, the poxvirus is administered in a therapeutically effective amount, e.g., at an amount sufficient to treat the infectious disease and/or the symptom thereof. In embodiments, the disease is not a cancer.

In an aspect, a method for preventing an infectious disease and/or a symptom thereof in a subject is provided. The method includes administering to the subject a poxvirus. In embodiments, the poxvirus is administered in a therapeutically effective amount, e.g., at an amount sufficient to prevent the infectious disease and/or the symptom thereof. In embodiments, the disease is not a cancer.

In an aspect, a method for preventing cytokine storm in a subject is provided. The method includes administering to the subject a poxvirus. In embodiments, the poxvirus is administered in a therapeutically effective amount, e.g., at an amount sufficient to prevent the cytokine storm. In embodiments, the cytokine storm is caused by an infection/infectious disease.

In an aspect, a method for treating a disease characterized by chronic inflammation is provided. The method includes administering to the subject a poxvirus and a stem cell, wherein the disease is not a cancer. In embodiments, the poxvirus is administered in a therapeutically effective amount, e.g., at an amount sufficient to treat the disease. In embodiments, the stem cell(s) is administered in a therapeutically effective amount, e.g., at an amount sufficient to treat the disease. In embodiments, the poxvirus and the stem cells are in the same (a single) composition.

In an aspect, a method for converting chronic inflammation into acute inflammation in a subject in need thereof is provided. The method includes administering to the subject a poxvirus, wherein the disease is not a cancer. In embodiments, the poxvirus is administered in a therapeutically effective amount, e.g., at an amount sufficient to convert the chronic inflammation to acute inflammation. In embodiments, the method further includes treating the acute inflammation. In embodiments, treating the acute inflammation includes administering to the subject a known treatment for acute inflammation.

In an aspect, a composition including a stem cell and optionally a poxvirus is provided, wherein the poxvirus includes a recombinant polynucleotide, wherein the recombinant polynucleotide encodes a therapeutic molecule. In embodiments, the therapeutic molecule treats the chronic inflammatory disease. In embodiments, the therapeutic molecule is an anti-inflammatory molecule.

In embodiments, the disease is a chronic inflammatory disease. In embodiments, the chronic inflammatory disease is an autoimmune disease. In embodiments, the chronic inflammatory disease is asthma, chronic peptic ulcer, tuberculosis, arthritis, periodontitis, ulcerative colitis, Crohn's disease, sinusitis, active hepatitis, atherosclerosis, dermatitis, inflammatory bowel disease (IBS), systemic lupus, fibromyalgia, Type 1 diabetes, psoriasis, Multiple sclerosis, Addison's disease, Grave's disease, Sjögren's syndrome, Hashimoto's thyroiditis, Myasthenia gravis, vasculitis, pernicious anemia, or celiac disease.

In embodiments, the inflammatory disease is transplant rejection, Dupytren's contracture, peyronies, periodontitis, endometriosis, hepatitis, glomerunephritis, atherscleroisis, cardiovascular disease, arthritis (e.g., osteoarthritis, rheumatoid arthritis, or psoriatic arthritis), inflammatory brain disease (including post-stroke, encephalitis), atherosclerosis, traumatic injury, infection, and/or shock. In an embodiment, the inflammatory disease is Chronic Obstructive Pulmonary Disease (COPD), such as emphysema, chronic bronchitis, or refractory (non-reversible) asthma.

In embodiments, the inflammatory disease is enteric fistula, chronic radiation damage (which causes inflammatory tissue defects such as radiation cystitis or radiation enteritis), duodenal ulcers, or a chronic inflammatory disease of the central nervous system, such as post stroke neuro-inflammation, schizophrenia, autism, addiction, chronic traumatic encephalopathy, or vaccine induced neuro-toxicity.

In an embodiment, the autoimmune disease is Myasthenia gravis (MG), Hashimoto's thyroiditis, vasculitis, Graves' disease, psoriasis, Chronic inflammatory demyelinating polyneuropathy (CIDP), Guillain barré syndrome, diabetes mellitus type 1, lupus, multiple sclerosis, rheumatoid arthritis, Addison's disease, Sjogren's syndrome, celiac disease, myositis, ankylosing spondylitis, or scleroderma.

In an embodiment, the infectious disease is caused by bacteria, virus, or fungus. In an embodiment, the infectious disease is caused by a virus. In an embodiment, the virus is a rhinovirus, coronavirus, influenza, or respiratory syncytial virus. In an embodiment, the coronavirus is severe acute respiratory syndrome coronavirus 2 (SARS-CoV-2).

In an embodiment, the infectious disease causes or is capable of causing cytokine storm in the subject. In an embodiment, the inflammatory disease causes or is capable of causing cytokine storm in the subject.

In embodiments, the stem cell includes a recombinant polynucleotide. In embodiments, the recombinant polynucleotide encodes a therapeutic molecule. In embodiments, the poxvirus includes a recombinant polynucleotide. In embodiments, the recombinant polynucleotide encodes a therapeutic molecule.

In embodiments, the therapeutic molecule treats the disease. In embodiments, the therapeutic molecule is a cytokine, a therapeutic antibody, a therapeutic fusion protein, an RNA, a peptide, or a polypeptide. In embodiments, the cytokine is an anti-inflammatory cytokine. In embodiments, the cytokine is selected from interleukin (IL)-1 receptor antagonist, IL-4, IL-6, IL-10, IL-11, IL-13, IFN-alpha, and transforming growth factor-beta. In embodiments, the therapeutic molecule is selected from abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), basiliximab (Simulect), daclizumab (Zinbryta), and muromonab (Orthoclone OKT3).

In embodiments, the therapeutic molecule improves treatment of the disease. For example, the therapeutic molecule may be a receptor that facilitates uptake of a therapeutic agent by a cell that expresses the therapeutic molecule. In another example, the therapeutic molecule may be an antigen recognized by a therapeutic agent. In another example, the therapeutic molecule may be an enzyme that is used by a cell to produce a therapeutic agent (e.g., a steroid). In embodiments, the therapeutic agent is an agent that treats the disease.

In embodiments, the method further includes administering a therapeutic agent to the subject. In embodiments, the therapeutic agent is in the same composition as the poxvirus and stem cell. In embodiments, the therapeutic agent is administered separately from the poxvirus and stem cell. In embodiments, the therapeutic agent is an agent that treats the disease. In embodiments, the therapeutic agent is selected from abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), basiliximab (Simulect), daclizumab (Zinbryta), and muromonab (Orthoclone OKT3).

In embodiments, the poxvirus and optionally the stem cell are administered to the subject by intravenous, intraperitoneal, intrathecal, intraventricular, intraarticular, intra-cerebro-ventricular, intrapleural, intra-parencymal, or intraocular injection. In embodiments, the poxvirus and optionally the stem cell are administered directly to a region affected by the disease. In embodiments, the poxvirus and optionally the stem cell are administered by guided delivery, e.g., MRI-guided delivery.

In embodiments, the stem cell is autologous to the subject. In embodiments, the stem cell is allogeneic to the subject. In embodiments, the subject is a human. In embodiments, the subject is a non-human animal. In embodiments, the subject is a domesticated animal. In embodiments, the subject is a companion animal.

In embodiments, the poxvirus is a vaccinia virus. In embodiments, the vaccinia virus is selected from Dryvax, ACAM1000, ACAM2000, Lister, EM63, LIVP, Tian Tan, Copenhagen, Western Reserve, Modified Vaccinia Ankara (MVA), New York City Board of Health, Dairen, Ikeda, LC16M8, Western Reserve Copenhagen, Tashkent, Tian Tan, Wyeth, IHD-J, and IHD-W, Brighton, Dairen I and Connaught strains. In embodiments, the vaccinia virus is ACAM1000 or ACAM2000. In embodiments, the vaccinia virus is a New York City Board of Health strain. In embodiments, the poxvirus is an attenuated virus.

In embodiments, the stem cell is selected from adult stem cell, embryonic stem cell, fetal stem cell, mesenchymal stem cell, neural stem cell, totipotent stem cell, pluripotent stem cell, multipotent stem cell, oligopotent stem cell, unipotent stem cell, adipose stromal cell, endothelial stem cell, induced pluripotent stem cell, bone marrow stem cell, cord blood stem cell, adult peripheral blood stem cell, myoblast stem cell, small juvenile stem cell, skin fibroblast stem cell, and combinations thereof. In embodiments, the stem cell is derived from a subject to be treated with the composition.

In embodiments, the stem cell is a modified stem cell. In embodiments, the modified stem cell expresses (was modified to express) a heterologous protein. In embodiments, the heterologous protein is a therapeutic molecule, a receptor that facilitates uptake of a therapeutic agent, an antigen that is recognized by a therapeutic agent, or an enzyme involved in production of a therapeutic agent. In embodiments, the therapeutic agent is an agent that treats the disease.

### DETAILED DESCRIPTION

After reading this description it will become apparent to one skilled in the art how to implement the invention in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present invention as set forth below.

Before the present invention is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description of the invention is divided into various sections only for the reader's convenience and disclosure found in any section may be combined with that in another section. Titles or subtitles may be used in the specification for the convenience of a reader, which are not intended to influence the scope of the present invention.

### I. Definitions

Unless defined otherwise, all technical and scientific terms used herein have the same meaning as commonly understood by one of ordinary skill in the art to which this invention belongs. In this specification and in the claims that follow, reference will be made to a number of terms that shall be defined to have the following meanings:

The terminology used herein is for the purpose of describing particular embodiments only and is not intended to be limiting of the invention. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to a dose amount means that the dose may vary by +/−10%.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

The terms “disease” or “condition” refer to a state of being or health status of a patient or subject capable of being treated with the compounds or methods provided herein.

The disease may be an autoimmune disease. The disease may be an inflammatory disease. The disease may be an infectious disease.

As used herein, the term “inflammatory disease” refers to a disease or condition characterized by aberrant inflammation (e.g. an increased level of inflammation compared to a control such as a healthy person not suffering from a disease). The term “chronic inflammatory disease” refers to an inflammatory disease that is persistent or recurring. Examples of chronic inflammatory diseases include autoimmune diseases, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, and atopic dermatitis.

In embodiments, the inflammatory disease is enteric fistula, chronic radiation damage (which causes inflammatory tissue defects such as radiation cystitis or radiation enteritis), duodenal ulcers, or a chronic inflammatory disease of the central nervous system, such as post stroke neuro-inflammation, schizophrenia, autism, addiction, chronic traumatic encephalopathy, or vaccine induced neuro-toxicity.

In embodiments, the inflammatory disease is transplant rejection, Dupytren's contracture, peyronies, periodontitis, endometriosis, hepatitis, glomerunephritis, atherscleroisis, cardiovascular disease, arthritis (e.g., osteoarthritis, rheumatoid arthritis, or psoriatic arthritis), inflammatory brain disease (including post-stroke, encephalitis), atherosclerosis, traumatic injury, infection, and/or shock. In an embodiment, the inflammatory disease is Chronic Obstructive Pulmonary Disease (COPD), such as emphysema, chronic bronchitis, or refractory (non-reversible) asthma.

As used herein, the term “autoimmune disease” refers to a disease or condition in which a subject's immune system has an aberrant immune response against a substance that does not normally elicit an immune response in a healthy subject. Examples of autoimmune diseases that may be treated with a compound, pharmaceutical composition, or method described herein include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Auto immune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

In embodiments, the inflammatory disease is enteric fistula, chronic radiation damage (which causes inflammatory tissue defects such as radiation cystitis or radiation enteritis), duodenal ulcers, or a chronic inflammatory disease of the central nervous system, such as post stroke neuro-inflammation, schizophrenia, autism, addiction, chronic traumatic encephalopathy, or vaccine induced neuro-toxicity.

In an embodiment, the autoimmune disease is Myasthenia gravis (MG), Hashimoto's thyroiditis, vasculitis, Graves' disease, psoriasis, Chronic inflammatory demyelinating polyneuropathy (CIDP), Guillain barré, diabetes mellitus type 1, lupus, multiple sclerosis, rheumatoid arthritis, Addison's disease, Sjogren's syndrome, celiac disease, myositis, ankylosing spondylitis, or scleroderma.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms (e.g., ocular pain, seeing halos around lights, red eye, very high intraocular pressure), fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is no prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

“Patient,” “subject,” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human. In embodiments, the human is a pediatric patient. In embodiments, a patient is a domesticated animal (e.g., goat, sheep, cow, horse, etc.). In embodiments, a patient is a companion animal, including but not limited to canine, feline, rodent (mouse, rat, gerbil, hamster, guinea pig, chinchilla, and the like), rabbit, ferret, etc.

An “effective amount” is an amount sufficient for a compound to accomplish a stated purpose relative to the absence of the compound (e.g. achieve the effect for which it is administered, treat a disease, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As is well known in the art, therapeutically effective amounts for use in humans can also be determined from animal models. For example, a dose for humans can be formulated to achieve a dose that has been found to be effective in animals. The dosage in humans can be adjusted by monitoring effectiveness and adjusting the dosage upwards or downwards, as described herein. Adjusting the dose to achieve maximal efficacy in humans based on the methods described herein and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the composition being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the composition. Thereafter, the dosage is increased by small increments until the optimum effect under circumstances is reached. Dosage amounts and intervals can be adjusted individually to provide levels of the administered composition effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intra-cerebro-ventricular, intrapleural, intra-parencymal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, etc. Administration also includes direct administration, e.g., directly to a site of inflammation. Direct administration may be via guided delivery, e.g., magnetic resonance imaging (MRI)-guided delivery. In embodiments, the administering does not include administration of any active agent other than the recited active agent.

“Co-administer” is meant that a composition described herein is administered at the same time, just prior to, or just after the administration of one or more additional therapies. The compositions provided herein can be administered alone or can be co-administered to the patient. Co-administration is meant to include simultaneous or sequential administration of the compositions individually or in combination (more than one composition). Thus, the preparations can also be combined, when desired, with other active substances.

A “stem cell” is a cell characterized by the ability of self-renewal through mitotic cell division and the potential to differentiate into a tissue or an organ. Among mammalian stem cells, embryonic stem cells (ES cells) and somatic stem cells (e.g., HSC, IPSCs) can be distinguished. Embryonic stem cells reside in the blastocyst and give rise to embryonic tissues, whereas somatic stem cells reside in adult tissues for the purpose of tissue regeneration and repair.

The term “infection” or “infectious disease” refers to a disease or condition that can be caused by organisms such as a bacterium, virus, fungi or any other pathogenic microbial agents. In embodiments, the infectious disease is caused by a pathogenic bacteria. Pathogenic bacteria are bacteria which cause diseases (e.g., in humans). In embodiments, the infectious disease is a bacteria associated disease (e.g., tuberculosis, which is caused by Mycobacterium tuberculosis). Non-limiting bacteria associated diseases include pneumonia, which may be caused by bacteria such as Streptococcus and Pseudomonas; or foodborne illnesses, which can be caused by bacteria such as Shigella, Campylobacter, and Salmonella. Bacteria associated diseases also includes tetanus, typhoid fever, diphtheria, syphilis, and leprosy. In embodiments, the infectious disease is Bacterial vaginosis (i.e. bacteria that change the vaginal microbiota caused by an overgrowth of bacteria that crowd out the Lactobacilli species that maintain healthy vaginal microbial populations) (e.g., yeast infection, or Trichomonas vaginalis); Bacterial meningitis (i.e. a bacterial inflammation of the meninges); Bacterial pneumonia (i.e. a bacterial infection of the lungs); Urinary tract infection; Bacterial gastroenteritis; or Bacterial skin infections (e.g. impetigo, or cellulitis). In embodiments, the infectious disease is a Campylobacter jejuni, Enterococcus faecalis, Haemophilus influenzae, Helicobacter pylori, Klebsiella pneumoniae, Legionella pneumophila, Neisseria gonorrhoeae, Neisseria meningitides, Staphylococcus aureus, Streptococcus pneumonia, or Vibrio cholera infection. In an embodiment, the infection is caused by a spriochete or borrelia, e.g. as related to lyme disease.

The terms “immune response” and the like refer, in the usual and customary sense, to a response by an organism that protects against disease. The response can be mounted by the innate immune system or by the adaptive immune system, as well known in the art.

The term “vaccine” as used herein, refers to any type of biological preparation contributing to or soliciting active immune responses against a particular disease or pathogen. Such biological preparation can include, but is not limited to, an antigen derived from a disease-causing agent or a portion of an antigen derived from a disease-causing agent. Such biological preparation can also be in the form of live attenuated preparation, including live, or weakened or modified disease causing agents or pathogens; or in the form of inactivated or killed disease-causing agents or pathogens. Alternative forms of such biological preparation further include, but are not limited to, the forms of subunit, toxoid, conjugate, DNA and recombinant vectors, or any suitable forms that might become developed or available in the future for soliciting active immune responses thereagainst.

It should be noted that in some embodiments, while the term “vaccine” is used herein, the vaccine need not provide significant immunity against smallpox (or any other pathogen), so long as it is effective against a disease as described herein. For example, the vaccine may be any immunogenic or infectious composition that treats the disease. In some cases, the term is used to identify certain materials or compositions, and not necessarily the ability of material or composition to provide immunity against smallpox, for example. The virus can be from any strain of virus, including for example, one or more listed below and elsewhere herein, including those that are not part of approved or contemplated vaccines.

As used herein, “virus” refers to any of a large group of entities referred to as viruses. Viruses typically contain a protein coat surrounding an RNA or DNA core of genetic material, but no semipermeable membrane, and are capable of growth and multiplication only in living cells. Viruses for use in the methods provided herein include, but are not limited, to a poxvirus, adenovirus, herpes simplex virus, Newcastle disease virus, vesicular stomatitis virus, mumps virus, influenza virus, measles virus, reovirus, human immunodeficiency virus (HIV), hanta virus, myxoma virus, cytomegalovirus (CMV), lentivirus, and any plant or insect virus.

As used herein, “heterologous nucleic acid” refers to a nucleic acid, DNA or RNA, which has been introduced into a virus or a cell (or the cell's ancestor). Such heterologous nucleic acid may comprise the sequence and operable regulatory elements for genes. For example, the heterologous nucleic acid may comprise a selection marker gene, a suicide gene, or a gene expressing a useful protein product that is not expressed endogenously, or expressed endogenously at low levels.

As used herein, the term “concurrently” as referring to administration of a poxvirus and a cell, refers to administration within 48 hours of each other. In some embodiments, the poxvirus and cell are administered within 36 hours of each other, within 24 hours of each other, within 12 hours of each other, within 10 hours of each other, within 8 hours of each other, within 6 hours of each other, within 4 hours of each other, within two hours of each other, within 1 hour of each other.

The term “autologous,” “autologous cell” or “autologous transplantation” as used herein in relation to cell transplantation indicates that the donor and recipient of the cells is the same individual. The term “allogenic,” “allogenic cell” or “allogenic transplantation” as used herein in relation to cell transplantation indicates that the donor and recipient of the cells are different individuals of the same species.

The term “adipose tissue” as used herein refers to body fat tissue, which is a loose connective tissue composed mostly of adipocytes. In addition to adipocytes, adipose tissue contains the stromal vascular fraction (SVF) of cells including preadipocytes, fibroblasts, vascular endothelial cells, a variety of immune cells, as well as regenerative stem cells.

The term “adipose stem cell” or “adipose-derived adult stem cell (ADASC)” as used herein refers to stem cells isolatable from adipose tissues, which are pluripotent stem cells capable of differentiation into numerous cell types. Adipose stem cells are found to be equivalent, if not superior to, bone marrow stem cells in terms of cell differentiation potential, angiogenesis and anti-inflammatory effects.

“Pharmaceutically acceptable excipient” and “pharmaceutically acceptable carrier” refer to a substance that aids the administration of an active agent to and/or absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

### II. Poxvirus

Variola virus is the cause of smallpox. Unlike variola virus, vaccinia virus, does not normally cause systemic disease in immune-competent individuals and it has therefore been used as a live vaccine to immunize against smallpox. Smallpox has been eradicated as a natural disease due to successful worldwide vaccination with Vaccinia virus. Routine smallpox vaccination has been discontinued for many years, except for people at higher risk of poxvirus infections (e.g., laboratory workers). Although the United States discontinued routine childhood immunization against smallpox in 1972, the use of smallpox vaccine is generally considered safe for pediatric use.

Attenuated strains derived from a pathogenic virus can be used for the manufacturing of a live vaccine. Non-limiting examples of viral strains that have been used as a smallpox vaccine include but are not limited to the Lister (also known as Elstree), New York City Board of Health (“NYCBH strain”), Dairen, Ikeda, LC16M8, Western Reserve (WR), Copenhagen, Tashkent, Tian Tan, Wyeth, IHD-J, and IHD-W, Brighton, Ankara, MVA, Dairen I, LIPV, LC16MO, LIVP, WR 65-16, EM63, and Connaught strains. In some embodiments, the smallpox vaccine as disclosed herein is an attenuated New York City Board of Health (NYCBOH) strain of vaccinia virus. In some embodiments, the NYCBOH strain of vaccinia virus may be ATCC VR-118 or CJ-MVB-SPX.

In some embodiments, the smallpox vaccine is non-attenuated.

In some embodiments, the smallpox vaccine is selected from Dryvax, ACAM1000, ACAM2000, Lister, EM63, LIVP, Tian Tan, Copenhagen, Western Reserve, or Modified Vaccinia Ankara (MVA). In some embodiments, the smallpox vaccine is not deficient in any genes present in one or more of these strains.

In some embodiments, the smallpox vaccine is a replication competent virus. In some embodiments, the smallpox vaccine is replication deficient.

### III. Stem Cells

Viruses are subject to significant elimination and/or neutralization following systemic administration. Stem cells acts as vehicles for shielding the disclosed smallpox vaccines from the elements of the humoral and cellular immunity in the blood stream. See, e.g., U.S. Pat. No. 10,105,436, which is incorporated herein by reference in its entirety.

Accordingly, stem cells can be used as a vehicle for in vivo delivery of a poxvirus to a site of disease in a subject. In some embodiments, the poxvirus is mixed with the stem cells to avoid the immune system from clearing the virus before the virus reaches the desired site. Thus, in some embodiments, disclosed herein is a method for treating a disease in a subject, comprising administering to the subject a poxvirus concurrently with a stem cell. In some embodiments, the poxvirus does not comprise heterologous nucleic acid. In some embodiments, the method further comprises concurrently administering a genetically engineered virus to the subject. Some embodiments relate to methods of preparing the preparing the stem cell and poxvirus compositions prior to administration.

In some embodiments, the vehicle stem cell is an autologous stem cell. In others it is non-autologous or allogeneic.

In some embodiments, the stem cells are selected from adult stem cells, embryonic stem cells, fetal stem cells, mesenchymal stem cells, neural stem cells, totipotent stem cells, pluripotent stem cells, multipotent stem cells, oligopotent stem cells, unipotent stem cells, adipose stromal cells, endothelial stem cells, induced pluripotent stem cells, bone marrow stem cells, cord blood stem cells, adult peripheral blood stem cells, myoblast stem cells, small juvenile stem cells, skin fibroblast stem cells, and combinations thereof. In some embodiments, the modified stem cell is an umbilical cord-derived mesenchymal like cell. In some embodiments, the umbilical cord-derived mesenchymal-like cell is an IMMSTEM™ cell. In some embodiments, the stem cell is an adipose stromal cell. One or more of the above-listed cells can be specifically excluded from some embodied compositions and methods.

In some embodiments, the stem cells are modified. Particularly, in some embodiments, the modified stem cell is an adult stem cell (ASC). In some embodiments, the modified stem cell is transformed with a viral vector. In some embodiments, the modified stem cell is transformed with a lentivirus or retrovirus. In some embodiments, the modified stem cell is transformed with a recombinant virus. In some embodiments, the modified stem cell is transiently transfected with an artificial chromosome, virus or plasmid DNA. In some embodiments, the virus is an oncolytic virus. In some embodiments, the virus is a vaccinia virus. In some embodiments, the virus is a replication-competent oncolytic vaccinia virus (VACV). In some embodiments, the modified stem cell is capable of localizing to a site of disease in the subject. In some embodiments, the modified stem cell is autologous. In some embodiments, the modified stem cell is allogeneic.

IMMSTEM™ cells are umbilical cord-derived mesenchymal-like cells, which possess pluripotent differentiation capacity and are characterized by unique surface markers and growth factor production. IMMSTEM™ cells possess numerous advantages compared to other stem cell sources, including ease of collection, higher rate of proliferation, very low immunogenicity, and ability to differentiate into tissues representative of all three germ layer components. In comparison to other mesenchymal stem cell (MSC) subtypes, IMMSTEM™ cells have demonstrated upregulated anti-inflammatory and migratory capacity due to a “cytokine priming” step, which is performed prior to administration. IMMSTEM™ cells are generated from human umbilical cords, which are obtained from full term women immediately after delivery. To stimulate a stress response, the cells are cultured for about 48 hours with interferon gamma. In some embodiments, the culturing with IFN-gamma can be from 1 to 72 hours or any value or sub range therein.

In some embodiments, the stem cells are infected with the poxvirus. Without being bound by theory, it is believed that infection of the stem cells with the poxvirus allows for production of additional poxvirus by the cells that will be targeted to the affected region.

In some embodiments, stem cells are not infected with the poxvirus. In embodiments, the stem cells that are not infected with the poxvirus have been engineered to express a therapeutic molecule. In embodiments, stem cells that are infected with the poxvirus and stem cells that are not infected and that have been engineered to express a therapeutic molecule are administered to the subject. Without being bound by theory, it is believed that the infected stem cells will target the poxvirus to the affected region, and the therapeutic molecule-expressing stem cells will target the therapeutic molecule to the affected region.

U.S. Pat. No. 10,105,436, which is incorporated herein by reference in its entirety, describes poxviruses, including smallpox vaccines, that can be used in the methods and compositions described herein.

### Adipose Stromal Vascular Fraction

Disclosed herein, in some embodiments, is a method of treating a disease in a subject, comprising administering to the subject a poxvirus concurrently with an adipose-derived stromal vascular fraction (SVF), wherein the disease is not a cancer. In some embodiments, the adipose-derived SVF is autologous. In some embodiments, the adipose-derived SVF is administered to the subject within about 24 hours of adipose tissue being taken from the subject. In some embodiments the adipose-derived SVF can be administered at any time after collection and up to about 48 hours post collection, or any time point or time sub range there between. In some embodiments, the smallpox vaccine is administered by intravenous, intraperitoneal, intrathecal, intraventricular, intraarticular, intra-cerebro-ventricular, intrapleural, intra-parencymal, or intraocular injection or intradermal injection, or any suitable methods delivering thereof.

Adipose tissue is an alternative to bone marrow as a source of stem cells, and provides a number of benefits, including: a) extraction of adipose derived cells is a simpler, less invasive procedure than bone marrow extraction; b) adipose tissue contains a higher content of mesenchymal stem cells (MSC) as compared to bone marrow; c) MSC from adipose tissue do not decrease in number with aging and can therefore serve as an autologous cell source for all patients; and d) adipose tissue is also a source of unique cell populations in addition to MSC that have therapeutic potential, including endothelial cells, regulatory T cells, and monocytes/macrophages.

MSC are poorly immunogenic and possess immune modulatory activity, features that are conserved among MSC from various tissues. This weak immunogenicity is believed to permit the survival and activity of allogeneic MSC when administered therapeutically.

SVF derived from whole lipoaspirate alleviates the need for extensive processing of the cells within, thereby minimizing the number of steps where contamination could be introduced (Kurita et al., Plast. Reconstr. Surg. 2008, 121:1033-1041; discussion 1042-1033; Yoshimura et al., Aesthetic Plast. Surg. 2008, 32:48-55; discussion 56-47). The safety of administration of adipose-derived cells is supported by autologous fat grafting, a common practice in cosmetic surgery (Hang-Fu et al., Aesthetic Plast. Surg. 1995, 19:427-437). Each of the aforementioned references is incorporated herein by reference in its entirety.

In some embodiments, the adipose-derived SVF is obtained by means and knowledge known to one of skill in the art. In some embodiments, the adipose-derived SVF is removed from the subject via a TIMEMACHINE™ device. In some embodiments, the adipose-derived SVF is removed from the subject with a 2.5 to 3 mM cannula. In some embodiments, one or more of the following devices can be utilized: PNC's Multi Station, CHA Biotech Cha-Station, Cytori Celution 800/CRS System, and Medi-Khan's Lipokit with MaxStem.

In some embodiments, the poxvirus and the adipose-derived SVF are administered concurrently. In some embodiments, the poxvirus and the adipose-derived SVF are administered simultaneously. In some embodiments, the poxvirus and the adipose-derived SVF are administered simultaneously through one administration vehicle. In some embodiments, the poxvirus and the adipose-derived SVF are administered simultaneously through one vessel, e.g. a syringe, via intravenous, intraperitoneal, intrathecal, intraventricular, intraarticular, intraocular, intra-cerebro-ventricular, intrapleural, intra-parencymal, or intradermal injection, or any suitable methods for delivery thereof.

U.S. Pat. No. 10,105,436, which is incorporated herein by reference in its entirety, describes stem cells and SVF, including methods of obtaining/making the same, that can be used in the methods and compositions described herein.

### IV. Methods of Use

### Inflammatory Disease

In one aspect, provided herein is a method for treating a chronic inflammatory disease in a subject in need thereof, the method comprising administering to the subject a poxvirus (e.g., smallpox, for example a smallpox vaccine) and a stem cell, wherein the disease is not a cancer. In embodiments, the chronic inflammatory inflammatory disease is an autoimmune disease. In embodiments, the chronic inflammatory inflammatory disease is asthma, chronic peptic ulcer, tuberculosis, arthritis, periodontitis, ulcerative colitis, Crohn's disease, sinusitis, active hepatitis, atherosclerosis, dermatitis, inflammatory bowel disease (IBS), systemic lupus, fibromyalgia, Type 1 diabetes, psoriasis, Multiple sclerosis, Addison's disease, Grave's disease, Sjögren's syndrome, Hashimoto's thyroiditis, Myasthenia gravis, vasculitis, pernicious anemia, celiac disease. In embodiments, the inflammatory disease is transplant rejection, Dupytren's contracture, peyronies, periodontitis, endometriosis, hepatitis, glomerunephritis, arthritis (e.g., osteoarthritis, rheumatoid arthritis, or psoriatic arthritis), inflammatory brain disease (including post-stroke, encephalitis), atherosclerosis. In an embodiment, the autoimmune disease is Myasthenia gravis (MG), Hashimoto's thyroiditis, vasculitis, Graves' disease, psoriasis, Chronic inflammatory demyelinating polyneuropathy (CIDP), Guillain barré, diabetes mellitus type 1, lupus, multiple sclerosis, rheumatoid arthritis, Addison's disease, Sjogren's syndrome, celiac disease, myositis, ankylosing spondylitis, or scleroderma.

In embodiments, the inflammatory disease is enteric fistula, chronic radiation damage (which causes inflammatory tissue defects such as radiation cystitis or radiation enteritis), duodenal ulcers, or a chronic inflammatory disease of the central nervous system, such as post stroke neuro-inflammation, schizophrenia, autism, addiction, chronic traumatic encephalopathy, or vaccine induced neuro-toxicity.

In embodiments, the chronic inflammatory disease is transplant rejection, Dupytren's contracture, peyronies, periodontitis, endometriosis, hepatitis, glomerunephritis, atherscleroisis, cardiovascular disease, arthritis (e.g., osteoarthritis, rheumatoid arthritis, or psoriatic arthritis), inflammatory brain disease (including post-stroke, encephalitis), atherosclerosis, traumatic injury, infection, and/or shock. In an embodiment, the inflammatory disease is Chronic Obstructive Pulmonary Disease (COPD), such as emphysema, chronic bronchitis, or refractory (non-reversible) asthma.

In an embodiment, the autoimmune disease is Myasthenia gravis (MG), Hashimoto's thyroiditis, vasculitis, Graves' disease, psoriasis, Chronic inflammatory demyelinating polyneuropathy (CIDP), Guillain barre, diabetes mellitus type 1, lupus, multiple sclerosis, rheumatoid arthritis, Addison's disease, Sjogren's syndrome, celiac disease, myositis, ankylosing spondylitis, or scleroderma.

In embodiments, the inflammatory disease is enteric fistula, chronic radiation damage (which causes inflammatory tissue defects such as radiation cystitis or radiation enteritis), duodenal ulcers, or a chronic inflammatory disease of the central nervous system, such as post stroke neuro-inflammation, schizophrenia, autism, addiction, chronic traumatic encephalopathy, or vaccine induced neuro-toxicity.

In embodiments, the disease is an infectious disease, traumatic injury, and/or shock.

In embodiments, a therapeutic molecule (therapeutic agent) is administered to the subject. Preferably, the therapeutic molecule (therapeutic agent) treats the disease. The therapeutic molecule (therapeutic agent) may be administered as part of the poxvirus/stem cell composition and/or separately. Where the therapeutic molecule (therapeutic agent) is administered as part of the poxvirus/stem cell composition, the therapeutic molecule (therapeutic agent) may be a separate component of the composition. Alternatively (or in addition), the therapeutic molecule may be expressed by the stem cell and/or encoded by the poxvirus.

In embodiments, the therapeutic molecule (therapeutic agent) is a therapeutic agent listed in Table 1:

TABLE 1
Chronic Inflammatory Diseases and Therapeutic Agents

| Disease | Peptide Therapeutic Agents | Other Therapeutic Agents |
|---|---|---|
| Ulcerative colitis | anti-TNF antibody therapy, | 5-aminosalicylates, |
| | golimumab, infliximab, | corticosteroids, azathioprine, |
| | adalimumab, vedolizumab | mercaptopurine, cyclosporine |
| COPD | | bronchodilators, inhaled steroids, |
| | | roflumilast |
| Atherscleroisis and | heparin | Statins, fibrates, beta blockers, |
| cardiovascular disease | | ACE inhibitors, Calcium channel |
| | | blockers, diuretics, aspirin, |
| Transplant rejection | Polyclonal antibodies, | Immunosuppressants |
| | monoclonal antibodies, T-cell | |
| | receptor directed antibodies, | |
| | IL-2 receptor directed | |
| | antibodies | |
| Peyronie's | Interferon | Calcium channel blockers, |
| | | Collagenase, Verapamil |
| Dupuytren's | | Needling, enzyme injections, |
| | | surgery |
| Periodontitis | | Topical antiseptics, penicillin, |
| | | antibiotics |
| Endometriosis | | Hormones |
| Hepatitis | | Hepatitis A vaccine, Hepatitis B |
| | | vaccine, antiviral drugs, liver |
| | | transplant, corticosteroids |
| Glomerulonephritis | | Corticosteroids, |
| | | immunosuppressants, |
| | | cyclophosphamide |
| Arthritis- osteo and | | NSAIDs, steroids, topical |
| rheumatoid and psoriatic | | analgesics, narcotics, |
| | | immunosuppressants |
| Autoimmune-MG, | | Immunosuppressants, calcium |
| Hashimotos, vasculitis, | | channel blockers, steroids, |
| graves psoriasis, CIDP, | | proton pump inhibitors, |
| Guillain barre, DM 1, | | Hormones |
| lupus, lupus, MS, RA, | | |
| Addison, sjogren's celiac, | | |
| myositis, ankylosing | | |
| spondylitis, scleroderma. | | |
| Inflammatory brain | | Antiviral drugs, anticonvulsants, |
| diseases including post | | antibiotics, blood thinners, |
| stroke and encephalitis | | statins, antihypertensive drugs |
| Atherosclerosis | | Statins, blood thinners |
| traumatic injury and | | Antibiotics, epinephrine, |
| infections cause shock, | | steroids, fluids |
| which is a result of the | | |
| inflammatory mediator | | |
| response | | |
| Interstitial cystitis | a protein that blocks APF*or | |
| | a protein that blocks aberrant | |
| | NF-κB signaling activation in | |
| | an abnormal phenotype | |

In embodiments, the therapeutic molecule (therapeutic agent) is selected from abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), basiliximab (Simulect), daclizumab (Zinbryta), and muromonab (Orthoclone OKT3).

In embodiments, the therapeutic molecule includes TGFβ, HGF, LIF, VEGF, EGF, BDNF, and/or NGF, or a fragment thereof.

In embodiments, the therapeutic molecule is an antibiotic. Antibiotics are well known in the art. The antibiotic may be any antibiotic. A skilled clinician can determine what antibiotic should be used based on the type of infection, as well as other standard determinations. Non-limiting examples of antibiotics are actinomycin, bacitracin, colistin, polymyxin B, gramicidins, polymyxins, bacitracins, glycopeptides, and the like.

In an aspect, a method for converting chronic inflammation into acute inflammation in a subject in need thereof is provided. The method includes administering to the subject a poxvirus, wherein the disease is not a cancer. In embodiments, the poxvirus is administered in a therapeutically effective amount, e.g., at an amount sufficient to convert the chronic inflammation to acute inflammation. In embodiments, the method further includes treating the acute inflammation. In embodiments, treating the acute inflammation includes administering to the subject a known treatment for acute inflammation.

In embodiments, the disease is interstitial cystitis. Without being bound by theory, it is believed that antiproliferative factor (APF) inhibits bladder cell proliferation by means of regulation of cell adhesion protein and growth factor production. In an embodiment, the therapeutic agent expressed by the virus and/or stem cell is a polypeptide that blocks APF expression and/or activity. In an embodiment, the therapeutic agent expressed by the virus and/or stem cell is a polypeptide that blocks NF-κB activity. In an embodiment, the therapeutic agent expressed by the virus and/or stem cell is a polypeptide that blocks aberrant NF-κB activity. In an embodiment, the therapeutic agent is HB-EGF or a fragment or variant thereof. See, e.g., Kim et al., BJU Int. 2009 February; 103(4): 541-546, which is incorporated herein by reference in its entirety.

In embodiments, the disease is atherosclerosis. Without being bound by theory, it is expected that induction of eNOS on the endothelial surface will mitigate atherosclerosis and may even reverse plaque formation. In an embodiment, the therapeutic agent expressed by the virus and/or stem cell is a polypeptide that induces eNOS.

In an embodiment, the infectious disease causes or is capable of causing cytokine storm in the subject. In an embodiment, administration of the poxvirus, and optionally the stem cell, treats cytokine storm.

### Infectious Disease

In an aspect, a method for treating an infectious disease in a subject in need thereof is provided. The method includes administering to the subject a poxvirus. In embodiments, the disease is not a cancer.

The infectious disease may be caused by any organism known to infect a subject. In embodiments, the infectious disease is caused by bacteria, virus, parasite, protozoa, helminths, or fungus. In embodiments, the infectious disease is caused by bacteria. In embodiments, the infectious disease is caused by a virus. In embodiments, the infectious disease is caused by a parasite. In embodiments, the infectious disease is caused by a fungus. In embodiments, the infectious disease may be caused by one or more of the agents listed in U.S. Pat. No. 8,715,677 and/or U.S. Patent Pub. No. 2019/0381160, each of which is incorporated herein by reference in its entirety.

In embodiments, the virus is a virus that infects humans. In embodiments, the virus causes respiratory infection, gastrointestinal infection, foodborne illness, skin infection, or sexually-transmitted infection. In embodiments, the virus is a rhinovirus, coronavirus, influenza, or respiratory syncytial virus. In embodiments, the virus is a coronavirus. In embodiments, the coronavirus is severe acute respiratory syndrome coronavirus 2 (SARS-CoV-2).

In embodiments, a stem cell is administered to the subject. The stem cell may be any type of stem cell, for example as described herein. In embodiments, the stem cell is a mesenchymal stem cell. In embodiments, the stem cell is an adipose stem cell.

In embodiments, a therapeutic molecule (therapeutic agent) is administered to the subject. Preferably, the therapeutic molecule (therapeutic agent) treats the disease or a symptom of the disease. The therapeutic molecule (therapeutic agent) may be administered as part of the poxvirus/stem cell composition and/or separately. Where the therapeutic molecule (therapeutic agent) is administered as part of the poxvirus/stem cell composition, the therapeutic molecule (therapeutic agent) may be a separate component of the composition. Alternatively (or in addition), the therapeutic molecule may be expressed by the stem cell and/or encoded by the poxvirus.

In embodiments, the therapeutic molecule is an antibiotic. Antibiotics are well known in the art. The antibiotic may be any antibiotic. A skilled clinician can determine what antibiotic should be used based on the type of infection, as well as other standard determinations. Non-limiting examples of antibiotics are actinomycin, bacitracin, colistin, polymyxin B, gramicidins, polymyxins, bacitracins, glycopeptides, and the like.

In embodiments, the therapeutic molecule is an anti-viral molecule. In embodiments, the therapeutic molecule is an antifungal molecule. In embodiments, the therapeutic molecule is an antiparasitic molecule. In embodiments, the therapeutic molecule is a vaccine. In embodiments, the therapeutic molecule includes a molecule having anti-inflammatory and/or trophic activity. In embodiments, the therapeutic molecule is TGFβ, HGF, LIF, VEGF, EGF, BDNF, and/or NGF, or a fragment thereof. In embodiments, the therapeutic molecule is a molecule that treats or prevents cytokine storm, e.g., leukaemia inhibitory factor (LIF).

In an embodiment, the infectious disease causes or is capable of causing cytokine storm in the subject. In an embodiment, administration of the poxvirus, and optionally the stem cell, treats cytokine storm.

### Cancer Patients

In an aspect, a method for treating or preventing an inflammatory disease or infectious disease in a subject having cancer is provided. Cancer patients are often immunocompromised, for example as a result of treatments for the cancer (chemotherapy, radiation, immunotherapy, and the like). Immunocompromised patients are more likely to be infected by a pathogen that causes an infectious disease.

In embodiments, the method includes administering to the subject a poxvirus. In embodiments, an inflammatory disease is treated. In embodiments, an inflammatory disease is prevented. In embodiments, an infectious disease is treated. In embodiments, an infectious disease is prevented. In embodiments, a stem cell is administered. Any method or composition described herein can be used to treat the subject.

Without being bound by theory, it is believed that administration of a poxvirus, optionally with a stem cell, as described herein will result in poxvirus infection of tumor cells in the patient. In embodiments, the poxvirus encodes a therapeutic molecule. In embodiments, the stem cells express a therapeutic molecule. In embodiments, the therapeutic molecule treats or prevents the inflammatory disease. In embodiments, the therapeutic molecule treats or prevents the infectious disease. In embodiments, the therapeutic molecule is a vaccine for the disease. In embodiments, the therapeutic molecule is an antibiotic. In embodiments, the therapeutic molecule is an anti-viral molecule. In embodiments, the therapeutic molecule is an antifungal molecule. In embodiments, the therapeutic molecule is an antiparasitic molecule.

In embodiments, infection of the tumor cells with the poxvirus results in expression of the therapeutic molecule by the tumor cells. Without being bound by theory, it is believed that expression of the therapeutic molecule by the tumor cells will result in an increase in the amount of therapeutic molecule delivered to the subject, and allow treatment or prevention of the disease.

### Administration

In embodiments, the poxvirus and/or the stem cell are administered to the subject by intravenous, intraperitoneal, intrathecal, intraventricular, intraarticular, intra-cerebro-ventricular, intrapleural, intra-parencymal, or intraocular injection. In embodiments, the poxvirus and optionally the stem cell are administered directly to a region affected by the disease. In embodiments, the poxvirus and optionally the stem cell are administered by direct injection. In embodiments, the poxvirus and optionally the stem cell are administered by MRI-guided delivery.

In embodiments, the stem cell is autologous to the subject. That is, the stem cell is derived from the patient to be treated. For example, an adipose-derived stromal vascular fraction may be taken from the patient and stem cells harvested or otherwise derived therefrom.

In embodiments, the stem cell is allogeneic to the subject. In embodiments, allogeneic stem cells are derived from subjects other than the patient. In embodiments, allogeneic stem cells are derived from cell lines.

In embodiments, the subject is a human.

In some embodiments, the poxvirus and the stem cell are administered simultaneously. In some embodiments, the poxvirus and the stem cell are administered simultaneously through one administration vehicle. In some embodiments, the poxvirus and the stem cell are administered simultaneously through one vessel, e.g. a syringe, via intravenous, intraperitoneal, intrathecal, intraventricular, intraarticular, intraocular, intra-cerebro-ventricular, intrapleural, intra-parencymal, or intradermal injection, or any suitable methods for delivery thereof.

The effective dosage of each of the treatment modalities disclosed herein may vary depending on various factors, including but not limited to the particular treatment, compound or pharmaceutical composition employed, the mode of administration, the condition being treated, and/or the severity of the condition being treated. Thus, the dosage regimen of the combination of the invention is selected in accordance with a variety of factors including the route of administration and the renal and hepatic function of the patient. A physician, clinician or veterinarian of ordinary skill can readily determine and prescribe the effective amount of the single active ingredients required to prevent, counter or arrest the progress of the condition. Optimal precision in achieving concentration of the active ingredients within the range that yields efficacy without toxicity requires a regimen based on the kinetics of the active ingredients' availability to target sites.

The amount of poxvirus administered to an average-sized adult can be, for example, 1×10² to 1×10¹⁰ plaque-forming units, 1×10³ to 1×10⁸ plaque-forming units, 1×10⁴ to 1×10⁶ plaque-forming units, or any value or sub range there between. As a specific example, about 2.5×10⁵ plaque-forming units can be used.

It should be understood that the embodiments described herein are not limited to vaccinations or vaccinating per se, but also relate to generating an immune response or reaction to an antigen associated with a disease. While the words “vaccine,” “vaccination,” or other like terms are used for convenience, it should be understood that such embodiments also relate to immune compositions, immunogenic compositions, immune response generation, immunization, etc., where absolute prophylactic immunity is not required or generated. For example, the embodiments referring to vaccination also can relate to generating or to assisting in creating an immunogenic or immune response against an antigen, regardless of whether that response results in absolute eradication or immunization against the disease to be treated.

U.S. Pat. No. 10,105,436, which is incorporated herein by reference in its entirety, describes methods of administering, making, storing, and using compositions comprising poxvirus and stem cells that can be used in the methods and compositions described herein.

### V. Compositions

In one aspect, provided herein is a composition comprising a poxvirus (e.g., smallpox, for example a smallpox vaccine) and optionally a stem cell, wherein the poxvirus comprises a recombinant polynucleotide, wherein said recombinant polynucleotide encodes a therapeutic molecule.

In one aspect, provided herein is a composition comprising a poxvirus (e.g., smallpox, for example a smallpox vaccine) and a stem cell, wherein the stem cell comprises a recombinant polynucleotide, wherein said recombinant polynucleotide encodes a therapeutic molecule.

In embodiments, the therapeutic molecule treats a chronic inflammatory disease. In embodiments, the therapeutic molecule treats an autoimmune disease. In embodiments, the therapeutic molecule treats an infectious disease, a traumatic injury, or shock. In embodiments, the therapeutic molecule is a cytokine, a therapeutic antibody, a therapeutic fusion protein, an antibiotic, an RNA, a nucleotide, a peptide, or a polypeptide. In embodiments, the cytokine is selected from interleukin (IL)-1 receptor antagonist, IL-4, IL-6, IL-10, IL-11, IL-13, IFN-alpha, and transforming growth factor-beta.

In one aspect, provided herein is a composition comprising a poxvirus (e.g., smallpox, for example a smallpox vaccine), optionally a stem cell, and a therapeutic agent. In an embodiment, the poxvirus comprises a recombinant polynucleotide, wherein said recombinant polynucleotide encodes a therapeutic molecule. In an embodiment, the stem cell comprises a recombinant polynucleotide, wherein said recombinant polynucleotide encodes a therapeutic molecule.

In embodiments, the therapeutic agent is a therapeutic agent listed in Table 1. In embodiments, the therapeutic agent is abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), basiliximab (Simulect), daclizumab (Zinbryta), or muromonab (Orthoclone OKT3).

In embodiments, the RNA is an antisense RNA, an siRNA, an RNA vaccine, miRNA, or RNA interference (RNAi).

Antibiotics are well known in the art. A skilled clinician can determine what antibiotic should be used based on the type of infection, as well as other standard determinations.

The stem cell may be any stem cell, in particular a stem cell as described herein.

The poxvirus may be any poxvirus, in particular a stem cell as described herein. In embodiments, the poxvirus is an attenuated virus.

Methods of preparing pharmaceutical compositions comprising the relevant treatments disclosed herein are known in the art and will be apparent from the art, from known standard references, such as Remington's Pharmaceutical Sciences, Mack Publishing Company, Easton, Pa., 18th edition (1990), which is incorporated herein by reference in its entirety.

In some embodiments, the compositions disclosed herein comprise a pharmaceutically acceptable carrier. As used herein, the term “pharmaceutically acceptable carrier” refers to solvents, diluents, preservatives, dispersion or suspension aids, isotonic agents, thickening or emulsifying agents, solid binders, and lubricants, appropriate for the particular dosage form. The skilled artisan is aware of a variety of different carriers that may be used in formulating pharmaceutical compositions and knows techniques for the preparation thereof (See Remington's Pharmaceutical Sciences Ed. by Gennaro, Mack Publishing, Easton, Pa., 1995; which is incorporated herein in its entirety by reference). The pharmaceutically acceptable carriers may include, but are not limited to Ringer's solution, isotonic saline, starches, potato starch, sugars, glucose, powdered tragacant, malt, gelatin, talc, cellulose and its derivatives, ethyl cellulose, sodium carboxymethyl cellulose, cellulose acetate excipients, cocoa butter, suppository waxes, agar, alginic acid, oils, cottonseed oil, peanut oil, safflower oil, sesame oil, olive oil, soybean oil, corn oil, glycols, propylene glycol, esters, ethyl laurate, ethyl oleate, buffering agents, aluminum hydroxide, magnesium hydroxide, phosphate buffer solutions, pyrogen-free water, ethyl alcohol, other non-toxic compatible lubricants, sodium lauryl sulfate, magnesium stearate, coloring agents, releasing agents, coating agents, sweetening, flavoring and perfuming agents. Pharmaceutically acceptable carriers may also include preservatives and antioxidants. One or more of the above-mentioned materials can be specifically excluded from the compositions and methods of some embodiments.

A composition disclosed herein comprising a poxvirus may comprise an adjuvant. Optionally, one or more compounds having adjuvant activity may be included in the composition. Adjuvants are non-specific stimulators of the immune system that enhance the host's immune response to the vaccine. Examples of adjuvants known in the art are Freund's Complete and Incomplete adjuvant, vitamin E, non-ionic block polymers, muramyldipeptides, ISCOMs (immune stimulating complexes), saponins, mineral oil, vegetable oil, and Carbopol. Adjuvants, especially suitable for mucosal application are, for example, E. coli heat-labile toxin (LT) or Cholera toxin (CT). Other suitable adjuvants are for example aluminum hydroxide, aluminum phosphate or aluminum oxide, oil-emulsions (e.g., of Bayol F® or Marcol 52®, saponins or vitamin-E solubilisate). One or more of the above-mentioned materials can be specifically excluded from the compositions and methods of some embodiments.

U.S. Pat. No. 10,105,436, which is incorporated herein by reference in its entirety, describes methods of administering, making, storing, and using compositions comprising poxvirus and stem cells that can be used in the methods and compositions described herein.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Preparation of ACAM2000 Vaccine

The vaccine vial should be removed from cold storage and brought to room temperature before reconstitution. The flip cap seals of the vaccine and diluent vials are removed, and each rubber stopper is wiped with an isopropyl alcohol swab and allowed to dry thoroughly.

Using aseptic technique and a sterile 1 mL syringe fitted with a 25 gauge×⅝″ needle, 0.3 mL of diluent is transferred to the vaccine vial. Vial is gently swirled to mix, preferably without getting product on the rubber stopper. The reconstituted vaccine should be a clear to slightly hazy, colorless to straw-colored liquid free from extraneous matter. Reconstituted vaccine is inspected visually for particulate matter and discoloration prior to administration. If particulate matter or discoloration is observed, the vaccine is not used and the vial is disposed safely.

After reconstitution of the lyophilized preparation, each vial contains approximately 2.5-12.5×10⁷ plaque-forming units (pfu) of live vaccinia virus. After reconstitution, ACAM2000 vaccine may be used within 6 to 8 hours if kept at room temperature (20-25° C., 68-77° F.). Unused, reconstituted ACAM2000 vaccine may be stored in a refrigerator (2-8° C., 36-46° F.) up to 30 days, after which it should be discarded as a biohazardous material. Personnel preparing and administering the vaccine should wear surgical or protective gloves and avoid contact of vaccine with skin, eyes or mucous membranes. The vaccine vial, its stopper, the diluent syringe, the vented needle used for reconstitution, the needle used for administration, and any material that came in contact with the vaccine should be discarded in leak-proof, puncture-proof biohazard containers. These containers should then be disposed of appropriately.

For vaccine application, the vaccine vial is gently swirled to mix, preferably without getting product on the rubber stopper. Using aseptic technique and a sterile 1 mL syringe fitted with a 25 gauge×⅝ in. needle, the entire contents of the vial are transferred to a labeled 20 cc syringe containing the SVF fraction. SVF-vaccine mixture is gently swirled to mix well and incubated at 37° C. for 2 to 4 hours.

### Example 2: Retrieval and Preparation of Adipose Stromal Vascular Fraction

The patient receives local anesthesia consisting of lidocaine 0.5% with epinephrine (1:400,000) with HCO₃ (8.4%) titrated to pH of 7.4 (generally 5 cc of HCO₃ in 60 cc total volume) and undergoes sterile preparation. The patient then undergoes a liposuction procedure utilizing, e.g., the TIME-MACHINE™ device, fat processing unit (syringe) and 2.5-3 mm cannula. Bacitracin ointment and a band aid are secured over the wound along with a compressive bandage.

The SVF (ADSCs) are prepared in a closed system according to the following protocol:
a. TIME-MACHINE® harvest of fat into 60 cc single use sterile fat processing syringe
b. Centrifuge the fat at 2800 rpm for 3 min.
c. Remove free fatty acids and debris (local/blood) via TP-109 closed system
d. Transfer 25 cc of condensed fat to SVF processing syringe
e. Add pre-warmed (38° C.) 25 cc of T-MAX® Time Machine Accelerator (GMP grade collagenase; Roche) containing 12.5 Wunsch units.
f. Incubate at 38° C. for 30-45 minutes.
g. Centrifuge at 200×g for 4 minutes
h. Remove supernatant fluid except for bottom 3 to 10 cc.
i. Add 50 cc DSLR as a washing solution to remove collagenase residue and centrifuge at 200×g for 4 minutes.
j. Repeat 2 more times for a total of 3 washings.
k. Remove all supernatant fluid leaving 3 to 10 cc of pellet collection—this is the Stromal Vascular Fraction.
l. Filter SVF through 100-micron filters to 20 cc syringe.
m. SVF sample collected and identified for number of cells, viability and to confirm no clumping or debris.
n. Aliquots of each cell suspension will be set aside for endotoxin testing and sterility staining. SVF will only be released for injection after confirmation of endotoxin assay results of less than or equal to 5 EU/kg/hr and negative gram stain results.
o. Cells will be resuspended in 20 ml of normal saline. Cell suspensions will be drawn into a syringe through an 18-gauge needle for injection. Up to 100 million viable cells will be used for injection.
p. The syringe will then be placed in a sealed specimen bag labeled with the patient's name and medical record number for transport to the procedure room for injection.

### Example 3: Administration of Adipose SVF with Smallpox Vaccine

### Deployment Methods:

Intravenous: the non-expanded, autologous stromal vascular fraction (SVF) extracted from up to 500 ml of lipoaspirate and purified by collagenase digestion and a series of washing steps and containing up to 100 million cells is incubated with vaccinia virus and will be delivered by intravenous injection in a volume of 20 mL.

What is claimed is:

1. A method for treating a chronic inflammatory disease in a subject in need thereof, the method comprising administering to the subject a poxvirus, wherein the disease is not a cancer.

2. A method for treating a disease characterized by chronic inflammation in a subject in need thereof, the method comprising administering to the subject a poxvirus, wherein the disease is not a cancer.

3. A method for converting chronic inflammation into acute inflammation, the method comprising administering to the subject a poxvirus, wherein the disease is not a cancer.

4. A method for treating an infectious disease in a subject in need thereof, the method comprising administering to the subject a poxvirus, wherein the disease is not a cancer.

5. A method for treating cytokine storm in a subject in need thereof, the method comprising administering to the subject a poxvirus, wherein the disease is not a cancer.

6. The method of claim 4, wherein the subject has an inflammatory disease.

7. The method of claim 4, wherein the subject has an infectious disease.

8. A method for treating or preventing an inflammatory disease or infectious disease in a subject having cancer, the method comprising administering to the subject a poxvirus.

9. The method of any one of claims 1 to 7, wherein a stem cell is administered with the poxvirus.

10. The method of any one of claim 1 to 2, 7 or 8, wherein the chronic inflammatory disease is an autoimmune disease.

11. The method of any one of claims 1 to 9, wherein the chronic inflammatory disease is selected from asthma, chronic peptic ulcer, tuberculosis, arthritis, periodontitis, ulcerative colitis, Crohn's disease, sinusitis, active hepatitis, atherosclerosis, dermatitis, inflammatory bowel disease (IBS), systemic lupus, fibromyalgia, Type 1 diabetes, psoriasis, Multiple sclerosis, Addison's disease, Grave's disease, Sjögren's syndrome, Hashimoto's thyroiditis, Myasthenia gravis, vasculitis, pernicious anemia, or celiac disease.

12. The method of claim 9, wherein the autoimmune disease is Myasthenia gravis (MG), Hashimoto's thyroiditis, vasculitis, Graves' disease, psoriasis, Chronic inflammatory demyelinating polyneuropathy (CIDP), Guillain barré, diabetes mellitus type 1, lupus, multiple sclerosis, rheumatoid arthritis, Addison's disease, Sjogren's syndrome, celiac disease, myositis, ankylosing spondylitis, or scleroderma.

13. The method of any one of claims 1 to 9, wherein the chronic inflammatory disease is transplant rejection, Dupytren's contracture, peyronies, periodontitis, endometriosis, hepatitis, glomerunephritis, atherscleroisis, cardiovascular disease, arthritis (e.g., osteoarthritis, rheumatoid arthritis, or psoriatic arthritis), inflammatory brain disease (including post-stroke, encephalitis), atherosclerosis, traumatic injury, infection, Chronic Obstructive Pulmonary Disease (COPD), and/or shock.

14. The method of any one of claims 1 to 9, wherein the inflammatory disease is enteric fistula, chronic radiation damage (which causes inflammatory tissue defects such as radiation cystitis or radiation enteritis), duodenal ulcers, or a chronic inflammatory disease of the central nervous system, such as post stroke neuro-inflammation, schizophrenia, autism, addiction, chronic traumatic encephalopathy, or vaccine induced neuro-toxicity

15. The method of claim 12, wherein the COPD is emphysema, chronic bronchitis, or refractory (non-reversible) asthma.

16. The method of any one of claims 3 to 8, wherein the infectious disease is caused by bacteria, virus, or fungus.

17. The method of claim 15, wherein the infectious disease is caused by a virus.

18. The method of claim 16, wherein the virus is a rhinovirus, coronavirus, influenza, or respiratory syncytial virus.

19. The method of claim 18, wherein the coronavirus is severe acute respiratory syndrome coronavirus 2 (SARS-CoV-2).

20. The method of any one of claims 1-19, wherein the poxvirus is a vaccinia virus.

21. The method of claim 20, wherein the vaccinia virus is selected from Dryvax, ACAM1000, ACAM2000, Lister, EM63, LIVP, Tian Tan, Copenhagen, Western Reserve, Modified Vaccinia Ankara (MVA), New York City Board of Health, Dairen, Ikeda, LC16M8, Western Reserve Copenhagen, Tashkent, Tian Tan, Wyeth, IHD-J, and IHD-W, Brighton, Dairen I and Connaught strains.

22. The method of claim 21, wherein the vaccinia virus is ACAM1000 or ACAM2000.

23. The method of claim 21, wherein the vaccinia virus is a New York City Board of Health strain.

24. The method of any one of claims 1-22, wherein the poxvirus is an attenuated virus.

25. The method of any one of claims 1-24, further comprising administering a stem cell to the subject.

26. The method of claim 25, wherein the stem cell comprises a recombinant polynucleotide, wherein said recombinant polynucleotide encodes a therapeutic molecule.

27. The method of any one of claims 1-26, wherein the poxvirus comprises a recombinant polynucleotide, wherein said recombinant polynucleotide encodes a therapeutic molecule.

28. The method of claim 26 or 27, wherein the therapeutic molecule treats the disease.

29. The method of any one of claims 26-28, wherein the therapeutic molecule is a cytokine, a therapeutic antibody, a therapeutic fusion protein, an antibiotic, an RNA, a receptor that facilitates uptake of a therapeutic agent, an antigen recognized by a therapeutic agent, or an enzyme that is used by a cell to produce a therapeutic agent.

30. The method of any one of claims 26-29, wherein the therapeutic molecule is selected from an anti-TNF antibody, a T-cell receptor directed antibody, IL-2 receptor directed antibody, and an interferon. The method of any one of the above claims, wherein the therapeutic molecule is selected from abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), basiliximab (Simulect), daclizumab (Zinbryta), and muromonab (Orthoclone OKT3).

31. The method of any one of the above claims, further comprising administering a therapeutic agent to the subject.

32. The method of claim 31, wherein the therapeutic agent is an agent that treats the chronic inflammatory disease.

33. The method of claim 31 or 32, wherein the therapeutic agent is selected from 5-aminosalicylates, corticosteroids, azathioprine, mercaptopurine, cyclosporine, bronchodilators, roflumilast, Statins, fibrates, beta blockers, ACE inhibitors, diuretics, aspirin, Calcium channel blockers, Collagenase, Verapamil, Topical antiseptics, penicillin, antibiotics, hormones, Hepatitis A vaccine, Hepatitis B vaccine, immunosuppressants, cyclophosphamide, NSAIDs, analgesics, narcotics, steroids, proton pump inhibitors, Antiviral drugs, anticonvulsants, blood thinners, antihypertensive drugs, and epinephrine.

34. The method of claim 31 or 32, wherein the therapeutic agent is selected from abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), basiliximab (Simulect), daclizumab (Zinbryta), and muromonab (Orthoclone OKT3).

35. The method of claim 31, wherein the therapeutic agent is an agent that treats a viral infection or a symptom thereof.

36. The method of any one of claims 25-35, wherein the stem cell is selected from adult stem cell, embryonic stem cell, fetal stem cell, mesenchymal stem cell, neural stem cell, totipotent stem cell, pluripotent stem cell, multipotent stem cell, oligopotent stem cell, unipotent stem cell, adipose stromal cell, endothelial stem cell, induced pluripotent stem cell, bone marrow stem cell, cord blood stem cell, adult peripheral blood stem cell, myoblast stem cell, small juvenile stem cell, skin fibroblast stem cell, and combinations thereof.

37. The method of claim 36, wherein the stem cell is an adipose stem cell.

38. The method of any one of claims 25-37, wherein the stem cell is a stem cell line or derived from a stem cell line.

39. The method of any one of claims 25-38, wherein the stem cell is a modified stem cell.

40. The method of claim 39, wherein the modified stem cell expresses a heterologous protein.

41. The method of claim 40, wherein the heterologous protein is a therapeutic molecule.

42. The method of claim 41, wherein the therapeutic molecule treats the disease.

43. The method of claim 41 or 42, wherein the therapeutic molecule is a cytokine, a therapeutic antibody, a therapeutic fusion protein, an antibiotic, an RNA, a receptor that facilitates uptake of a therapeutic agent, an antigen recognized by a therapeutic agent, or an enzyme that is used by a cell to produce a therapeutic agent.

44. The method of any one of claims 41-43, wherein the therapeutic molecule is selected from an anti-TNF antibody, a T-cell receptor directed antibody, IL-2 receptor directed antibody, and an interferon. The method of any one of the above claims, wherein the therapeutic molecule is selected from abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), basiliximab (Simulect), daclizumab (Zinbryta), and muromonab (Orthoclone OKT3).

45. The method of any one of claims 1-44, wherein the poxvirus and/or the stem cell are administered to the subject by intravenous, intraperitoneal, intrathecal, intra-cerebro-ventricular, intrapleural, intra-parencymal, intraventricular, intraarticular, or intraocular injection.

46. The method of any one of claims 1-44, wherein the poxvirus and/or the stem cell are administered directly to a region affected by the disease.

47. The method of claim 46, wherein the poxvirus and/or the stem cell are administered by MRI-guided delivery.

48. The method of any one of the above claims, wherein the stem cell is autologous to the subject.

49. The method of any one of the above claims, wherein the stem cell is allogeneic to the subject.

50. The method of any one of the above claims, wherein the subject is a human.

51. The method of any one of claims 1-49, wherein the subject is a domesticated animal.

52. The method of any one of claims 1-49, wherein the subject is a companion animal.

53. The method of claim 52, wherein the subject is a canine.

54. A composition comprising a stem cell and a poxvirus, wherein the poxvirus comprises a recombinant polynucleotide, wherein said recombinant polynucleotide encodes a therapeutic molecule.

55. The composition of claim 54, wherein the therapeutic molecule treats an inflammatory disease.

56. The composition of claim 54 or 55, wherein the therapeutic molecule is a cytokine, a therapeutic antibody, a therapeutic fusion protein, an RNA, an antibiotic, a receptor that facilitates uptake of a therapeutic agent, an antigen recognized by a therapeutic agent, or an enzyme that is used by a cell to produce a therapeutic agent.

57. The composition of claim 54 or 55 wherein the therapeutic molecule is selected from an anti-TNF antibody, a T-cell receptor directed antibody, IL-2 receptor directed antibody, and an interferon.

58. The composition of claim 54 or 55, wherein the therapeutic molecule is selected from abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), basiliximab (Simulect), daclizumab (Zinbryta), and muromonab (Orthoclone OKT3).

59. The composition of any one of claims 54-58, wherein the poxvirus is a vaccinia virus.

60. The composition of claim 59, wherein the vaccinia virus is selected from Dryvax, ACAM1000, ACAM2000, Lister, EM63, LIVP, Tian Tan, Copenhagen, Western Reserve, Modified Vaccinia Ankara (MVA), New York City Board of Health, Dairen, Ikeda, LC16M8, Western Reserve Copenhagen, Tashkent, Tian Tan, Wyeth, IHD-J, and IHD-W, Brighton, Dairen I and Connaught strains.

61. The composition of claim 60, wherein the vaccinia virus is ACAM1000 or ACAM2000.

62. The composition of claim 60, wherein the vaccinia virus is a New York City Board of Health strain.

63. The composition of any one of claims 54-62, wherein the poxvirus is an attenuated virus.

64. The composition of any one of claims 54-63, wherein the stem cell comprises a recombinant polynucleotide, wherein said recombinant polynucleotide encodes a second therapeutic molecule.

65. The composition of claim 64, wherein the second therapeutic molecule is selected from abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), basiliximab (Simulect), daclizumab (Zinbryta), and muromonab (Orthoclone OKT3).

66. The composition of any one of claims 54-65, wherein the stem cell is selected from adult stem cell, embryonic stem cell, fetal stem cell, mesenchymal stem cell, neural stem cell, totipotent stem cell, pluripotent stem cell, multipotent stem cell, oligopotent stem cell, unipotent stem cell, adipose stromal cell, endothelial stem cell, induced pluripotent stem cell, bone marrow stem cell, cord blood stem cell, adult peripheral blood stem cell, myoblast stem cell, small juvenile stem cell, skin fibroblast stem cell, and combinations thereof.

67. The composition of any one of claims 54-66, wherein the stem cell is a modified stem cell.

68. The composition of claim 67, wherein the modified stem cell expresses a heterologous protein.

69. The composition of any one of claims 54-68, wherein the stem cell is derived from a subject to be treated with the composition.

70. The composition of any one of claims 54-69, wherein the stem cell is derived from a human.

71. The composition of any one of claims 54-69, wherein the stem cell is derived from a domesticated animal.

72. The composition of any one of claims 54-69, wherein the stem cell is derived from a companion animal.

73. The composition of claim 72, wherein the stem cell is derived from a canine subject.

74. The composition of any one of claims 54 to 73, wherein the therapeutic molecule treats an inflammatory disease or an infectious disease.
